(19) United States
(12) Patent Application Publication
(10) Pub. No.: US 2016/0213680 A1
(43) Pub. Date: Jul. 28, 2016

(54) COMPOSITIONS AND METHODS USING FLUMAZENIL WITH OPIOID ANALGESICS FOR TREATING PAIN AND/OR ADDICTION, AND WITH DIVERSION AND/OR OVERDOSE MITIGATION

(71) Applicant: SOTERIAN PHARMACEUTICALS, LLC, Maitland, FL (US)

(72) Inventors: Christopher Seth Hassan, Maitland, FL (US); James Gregory Sullivan, Birmingham, AL (US)

(21) Appl. No.: 15/004,788

(22) Filed: Jan. 22, 2016

Related U.S. Application Data
(60) Provisional application No. 62/266,359, filed on Dec. 11, 2015.
(60) Provisional application No. 62/107,089, filed on Jan. 23, 2015.

(51) Int. Cl. A61K 31/5517; A61K 9/00; A61K 9/20; A61K 31/485; A61K 31/137

(52) U.S. Cl. CPC A61K 31/5517; A61K 31/485; A61K 31/137; A61K 9/2054; A61K 9/2027; A61K 9/2013; A61K 9/006; A61K 9/0053

(57) ABSTRACT

In some embodiments, the present invention may be directed to various pharmaceutical dosage units comprising at least one opioid analgesic in combination with a benzodiazepine blocker. In some embodiments, the present invention may be directed to various methods of treatment using such pharmaceutical dosage units. In some embodiments, these pharmaceutical dosage units may be used for treating: pain, chronic pain, sub-acute pain, chronic and sub-acute pain, anxiety, addiction in general, opiate addiction, and benzodiazepine addiction, opiate and benzodiazepine addiction; opiate dependence, benzodiazepine dependence; and for reducing a likelihood of overdose associated with concomitant use of opiates with benzodiazepines, and for mitigating against drug diversion. These pharmaceutical dosage units may be delivered by a variety of delivery methods and/or delivery systems, comprising one or more of: mucosal, sublingual, buccal, nasal inhalation, depot, implantable rod, transdermal patch, parenteral, intravenous, intrathecal, subcutaneous, intramuscular, and the like.

### PRIORITY NOTICE

The present application claims priority under Title 35 United States Code (USC) Section 119(e) to U.S. Provisional Patent Application Ser. No. 62/266,359 filed Dec. 11, 2015, and to U.S. Provisional Patent Application Ser. No. 62/107,089 filed Jan. 23, 2015, the disclosures of which are incorporated herein by reference in their entirety.

### TECHNICAL FIELD OF THE INVENTION

The present invention relates in general to pharmaceutical compositions and methods of treatment using pharmaceutical compositions and more specifically to pharmaceutical compositions comprising a benzodiazepine blocker in combination with an opioid analgesic, and wherein the methods of treatment using the pharmaceutical compositions may be for treating: pain, chronic pain, sub-acute pain, chronic and sub-acute pain, anxiety, addiction in general, opiate addiction, and benzodiazepine addiction, opiate and benzodiazepine addiction; opiate dependence, benzodiazepine dependence; and for reducing a likelihood of overdose (e.g., respiratory arrest) associated with concomitant use of opiates with benzodiazepines, and for mitigating against drug diversion, such as diversion of opioid analgesics and/or of benzodiazepines.

### COPYRIGHT AND TRADEMARK NOTICE

A portion of the disclosure of this patent application may contain material that is subject to copyright protection. The owner has no objection to the facsimile reproduction by anyone of the patent document or the patent disclosure, as it appears in the Patent and Trademark Office patent file or records, but otherwise reserves all copyrights whatsoever.

Certain marks referenced herein may be common law or registered trademarks of third parties affiliated or unaffiliated with the applicant or the assignee. Use of these marks is by way of example and should not be construed as descriptive or to limit the scope of this invention to material associated only with such marks.

### BACKGROUND OF THE INVENTION

Circa late 2014, approximately 81 million United States (US) residents live with chronic pain. Severe chronic pain has historically been treated with various opioid analgesics, e.g., morphine. For example, various opiates have been effectively used for treating acute, sub-acute and/or chronic pain for over 4 thousand years. Prior to 1980 opiate analgesics were primarily prescribed to patients suffering from cancer related pain. In the mid 1980's physicians began treating non-cancer pain with opiate analgesics.

The treatment of chronic pain is presently the most costly health care problem in the US. Estimated annual costs, including direct medical expenses, lost income, lost productivity, compensation payments, and legal charges are currently about $460 billion and the costs are rising. Estimates indicate that by 2030, in the US alone, 148 million people will have chronic pain conditions, and associated annual direct costs will rise to $798 billion. Thus, pain management has been identified as one of the most difficult challenges for the health care industry.

However, in humans, opiates, including opioid analgesics, frequently cause euphoria along with physical and psychological dependence resulting in opiate addiction. Opiate dependence and addiction is a growing epidemic in the US and is spreading around the world. As opposed to other forms of addiction, it has been demonstrated in multiple studies that opiate dependent patients have better outcomes and with less mortality when treated with medically assisted treatment rather than behavioral health treatment alone. Opiate dependence encompasses both prescription opiates as well as heroin. The total US societal costs of prescription opioid abuse were estimated at $55.7 billion in 2007. Workplace costs accounted for $25.6 billion (46 percent), health care costs accounted for $25.0 billion (45 percent), and criminal justice costs accounted for $5.1 billion (9 percent). Workplace costs were driven by lost earnings from premature death ($11.2 billion) and reduced compensation/lost employment ($7.9 billion). Health care costs consisted primarily of excess medical and prescription costs ($23.7 billion). Criminal justice costs were largely comprised of correctional facility ($2.3 billion) and police costs ($1.5 billion).

In a study published in Drug and Alcohol Dependence, the study documented the costs of heroin addiction in the US, both to the addict and society at large. Using a cost-of-illness approach, costs were estimated in four broad areas: medical care, lost productivity, crime, and social welfare. It was estimated that the cost of heroin addiction in the US was $21.9 billion. Of these costs, productivity losses accounted for $11.5 billion (53 percent), criminal activities $5.2 billion (24 percent), medical care $5.0 billion (23 percent), and social welfare $0.1 billion (0.5 percent). More recently, heroin use and addiction has dramatically increased and thus the associated costs have increased as well.

Since the mid 1980's overdose deaths, emergency room (ER) visits related to pain medicine use, especially various opioid analgesics, and drug rehab admissions have grown significantly each year. Deaths from overdose, in the US, are now more common than motor vehicle accidents or violent killings. Effective pain treatment is needed that is safer than conventional opiate pain medications. For example, buprenorphine is 30 times stronger than morphine for pain. However, buprenorphine has less addiction potential, less respiratory depression and is 100 times less likely to cause fatal overdose than opiates such as oxycodone or methadone.

Many drugs enhance the addictive and euphoric effects of opiates and are therefore used in combination regularly by those persons using opiates for recreation or purposes other than pain or purposes in addition to treating pain. This tends to compound the problem of opiate addiction and increases illegal diversion of opiates and/or of the drug being combined with the opiate use.

Various benzodiazepines are the most common drugs used by opiate addicted persons to enhance the euphoric effects of opiates. Many sedatives are in the benzodiazepine class of drugs.

At least some benzodiazepines may work by binding to an ionic control site, known as a GABA receptor, that may exist on substantially all normal human brain neurons. When benzodiazepines are taken, such brain nerves are made “electrically” much less reactive and simply “fall asleep.” When a benzodiazepine is combined with an opiate these effects are markedly enhanced and somnolence and death can occur; and typically with a greater likelihood than if the benzodiazepine or the opiate was used alone. That is why there is a box warning on opiates instructing patients to avoid taking benzodiazepines concurrently. Despite this, such warnings are often ignored and patients often take the two medications together and die by the thousands every year.

Opiates work through an entirely different mechanism. Opiate like chemicals are naturally produced by the brain to “steer” behavior. Those activities that are important to procreation and/or survival cause such natural opiate like chemicals, often known as “endorphins” to be produced and these chemicals directly stimulate pleasure, relaxation, and memory areas of the brain, making that activity more likely to occur again in the future. Benzodiazepines markedly enhance the relaxation effect of opiates and when taken together can make the human body unable to sense the need to breath and death may occur by respiratory arrest.

For example, opiates which may reduce respirations may include, but may not be limited to: bromadol, buprenorphine, carfentanil, clonitazene, codeine, dextropropoxyphene, diamorphine (heroin), dihydrocodeine, dihydroetorphine, etonitazene, etorphine, fentanyl, hydrocodone, hydromorphone, levorphanol, methadone, morphine, oxycodone, oxymorphone, pentazocine, pethidine (meperidine hydrochloride), propoxyphene, sufentanil, tapentadol, tramadol, and the like.

For example, benzodiazepines whose effect increases risk of overdose, including respiratory arrest, when taken concomitantly with opiates may include, but may not be limited to the following one or more benzodiazepines: alprazolam (e.g., Xanax), bretazenil, bromam, bromazepam, brotizolam, cinolazepam, chlordiazepoxide, clonazepam (e.g., Klonopin), clorazepate, clotiazepam, cloxazolam, delorazepam, diazepam (e.g., Valium), diclazepam, estazolam, etizolam, ethyl loflazepate, flubromazepam, flunitrazepam, flurazepam, flutoprazepam, halazepam, ketazolam, lectopam, lexaurin, lexotanil, lexotan, loprazolam, lorazepam (e.g., Ativan), lormetazepam, medazepam, midazolam, nimetazepam, nitrazepam, nordazepam, oxazepam, phenazepam, pinazepam, prazepam, premazepam, pyrazolam, quazepam, temazepam, tetrazepam, triazolam, and the like.

The most common and deadly trend in opiate abuse is the combination of an opiate with a benzodiazepine. This combination increases the intended effect of both drugs on the addict while providing a significant increase in overdose and mortality. Increasing the danger for the patient presenting to the emergency personnel, is the fact that in most cases concomitant benzodiazepine overdose is only considered when it is too late.

Over one half the overdose death cases in the US involve mixing two drugs and the most common “second drug” is some type of benzodiazepine. Deaths from the combination of benzodiazepines and opiates have increased by more than 10 percent each year since 1999. The use of benzodiazepines with opiates together is the leading cause of death in people (age under 52) in several states in the US and is growing significantly each year.

Statistics published by SAMSHA in 2012 illustrate this growing epidemic of combined opiate use with benzodiazepines. (Substance Abuse and Mental Health Services Administration (SAMSHA) is an arm of the US federal government's Department of Health and Human Services.) The number of substance abuse treatment admissions reporting both benzodiazepine and narcotic pain reliever abuse increased 569.7 percent from 5,032 admissions in 2000 to 33,701 admissions in 2010, while the number of all other admissions decreased by 9.6 percent during the same period. In the month prior to treatment admission, 57.1 percent of benzodiazepine and narcotic pain reliever combination admissions reported daily use of narcotic pain relievers and 45.5 percent reported daily use of benzodiazepines.

In an effort to mitigate against this problem, the FDA (Food and Drug Administration) has mandated prescribers information sheets for all opiates that contain warnings for doctors and patients to avoid taking opiates concurrently with sedatives (e.g., benzodiazepines).

In those combination overdose cases causing death, the two medications (e.g., an opiate and a benzodiazepine) were in most cases not prescribed to the patient that died and instead prescriptions were bought off the street or gotten from friends or family or prescribed by different doctors unaware of the other prescription. This is the problem of diversion, i.e., diverting what may have been a validly prescribed medication, finding its way into a non-prescribed use.

For example, many of the safety benefits of buprenorphine with respect to traditional opiates are canceled or defeated when benzodiazepines are co-administered with buprenorphine. This is particularly a problem because buprenorphine is presently heavily diverted. This means a person other than the intended patient that was actually prescribed the buprenorphine may end up using the buprenorphine. Quite frequently opiate addicts are also addicted or dependent on benzodiazepines such as Xanax (alprazolam), Ativan (lorazepam), Valium (diazepam), Klonopin (clonazepam), and the like.

There is a need in the art for pharmaceutical compositions comprising a benzodiazepine blocker (e.g., flumazenil) in combination with an opioid analgesic (e.g., buprenorphine), and wherein the methods of treatment using such pharmaceutical compositions may be for treating: pain, chronic pain, sub-acute pain, chronic and sub-acute pain, anxiety, addiction in general, opiate addiction, and benzodiazepine addiction, opiate and benzodiazepine addiction; opiate dependence, benzodiazepine dependence; and for reducing a likelihood of overdose associated with concomitant use of opiates with benzodiazepines, and for mitigating against illegal drug diversion, such as opioid analgesic diversion and/or benzodiazepine diversion.

By adding a benzodiazepine blocker to an opioid analgesic formulation it makes the resulting combination far less attractive to the general addict population and to illegal drug dealers, but the combination does not affect the opioid analgesic's efficacy for the prescribed use in treating pain (chronic and/or sub-acute) and/or in treating opiate addiction. And actually the combination of opioid analgesics with a benzodiazepine blocker may result in unexpected enhanced pain relief and an unexpected increase in wellbeing.

It is to these ends that the present invention has been developed.

### BRIEF SUMMARY OF THE INVENTION

To minimize the limitations in the prior art, and to minimize other limitations that will be apparent upon reading and understanding the present specification, the present invention may describe various pharmaceutical dosage units comprising at least one opioid analgesic in combination with a benzodiazepine blocker; as well as method of using such pharmaceutical dosage units. In some exemplary embodiments, the at least one opioid analgesic may be buprenorphine. In some exemplary embodiments, the benzodiazepine blocker may be flumazenil. In some embodiments, the buprenorphine or the at least one opioid analgesic may be present in the pharmaceutical dosage unit in the range of about 1 to 61 milligrams (mg) and the flumazenil may be present in the pharmaceutical dosage unit at about 0.15 to 61 mg.

In some embodiments, the present invention may be directed to various methods of treatment using such pharmaceutical dosage units on a human, such as human patient. In some embodiments, these pharmaceutical dosage units may be used for treating: pain, chronic pain, sub-acute pain, chronic and sub-acute pain, anxiety, addiction in general, opiate addiction, and benzodiazepine addiction, opiate and benzodiazepine addiction; opiate dependence, benzodiaze pine dependence; and for reducing a likelihood of overdose associated with concomitant use of opiates with benzodiazepines (such as mitigating against unintended over sedation and respiratory arrest), and for mitigating against illegal diversion, such as opioid analgesic diversion and/or benzodiazepine diversion.

These pharmaceutical dosage units may be delivered by a variety of delivery methods and/or delivery systems, comprising one or more of: mucosal, sublingual, buccal, nasal inhalation, depot, implantable rod, transdermal patch, parenteral, intravenous (IV), intrathecal, subcutaneous (SC), intramuscular (IM), and the like of delivery means.

It is an objective of the present invention to provide alternative pharmaceutical dosage units as well as methods of use and treatment utilizing the alternative pharmaceutical dosage units that may replace existing opioid analgesics for treating pain (chronic and sub-acute).

It is another objective of the present invention to provide alternative pharmaceutical dosage units as well as methods of treatment utilizing the alternative pharmaceutical dosage units that may replace existing opiates for treating and/or managing opiate addiction.

It is another objective of the present invention to provide alternative pharmaceutical dosage units as well as methods of treatment utilizing the alternative pharmaceutical dosage units that may replace existing opiates for treating and/or managing opiate addiction, and may replace existing opioid analgesics for treating pain wherein the alternative pharmaceutical dosage units and/or the methods of treatment utilizing the alternative pharmaceutical dosage units may comprise a benzodiazepine blocker, such that a likelihood overdose commonly associated with concomitant use of opiates with benzodiazepines may be reduced.

It is another objective of the present invention to provide alternative pharmaceutical dosage units as well as methods of treatment utilizing the alternative pharmaceutical dosage units that may replace existing opiates for treating and/or managing opiate addiction, and may replace existing opioid analgesics for treating pain wherein the alternative pharmaceutical dosage units and/or the methods of treatment utilizing the alternative pharmaceutical dosage units may comprise a benzodiazepine blocker, such that a likelihood of illegal opiate diversion may be reduced.

It is another objective of the present invention to provide alternative pharmaceutical dosage units as well as methods of treatment utilizing the alternative pharmaceutical dosage units that may replace existing opiates for treating and/or managing opiate addiction, and/or may replace existing opioid analgesics for treating pain wherein the alternative pharmaceutical dosage units and/or the methods of treatment utilizing the alternative pharmaceutical dosage units may be embraced by prescribing physicians concerned with concomitant use of opiates with benzodiazepines.

It is another objective of the present invention to provide alternative pharmaceutical dosage units as well as methods of treatment utilizing the alternative pharmaceutical dosage units that may permit prescribing physicians to switch (covert) from an existing pain treatment prescription to the alternative pharmaceutical dosage units with minimal opiate withdrawal issues.

It is yet another objective of the present invention to provide alternative pharmaceutical dosage units as well as methods of treatment utilizing the alternative pharmaceutical dosage units that may replace existing opiates for treating and/or managing opiate addiction, and may replace existing opioid analgesics for treating pain wherein the alternative pharmaceutical dosage units and/or the methods of treatment utilizing the alternative pharmaceutical dosage units may reduce the side effects and/or the severity of side effects typically associated with opiate addiction (opiate withdrawal).

These and other advantages and features of the present invention are described herein with specificity so as to make the present invention understandable to one of ordinary skill in the art, both with respect to how to practice the present invention and how to make the present invention.

### DETAILED DESCRIPTION OF THE INVENTION

In some embodiments, the present invention may be directed to various pharmaceutical dosage units comprising at least one opioid analgesic in combination with a benzodiazepine blocker. In some exemplary embodiments, the at least one opioid analgesic may be buprenorphine. In some exemplary embodiments, the benzodiazepine blocker may be flumazenil. In some embodiments, the buprenorphine or the at least one opioid analgesic may be present in a first therapeutically effective amount and the flumazenil may be present in a second therapeutically effective amount. The first and the second therapeutically effective amounts may vary according to a delivery method of administration. For example, and without limiting the scope of the present invention, the first therapeutically effective amount may seek to result in a target serum concentration of buprenorphine of 0.1 nanograms (ng) per milliliter (mL) to 11 ng/mL.

In some embodiments, the buprenorphine or the at least one opioid analgesic may be present in the pharmaceutical dosage unit in the range of about 0.1 to 61 milligrams (mg) and the flumazenil may be present in the pharmaceutical dosage unit at about 0.15 to 61 mg.

Unless otherwise noted, as used herein, “about” in reference to ranges may mean plus or minus 0.1 of the unit noted, e.g., plus or minus 0.1 mg.

In some embodiments, the present invention may be directed to various methods of treatment using such pharmaceutical dosage units to a human, such as a human patient. In some embodiments, these pharmaceutical dosage units may be used for treating: pain, chronic pain, sub-acute pain, chronic and sub-acute pain, anxiety, addiction in general, opiate addiction, and benzodiazepine addiction, opiate and benzodiazepine addiction; opiate dependence, benzodiazepine dependence; and for reducing a likelihood of overdose associated with concomitant use of opiates with benzodiazepines (e.g., mitigating against unintended over sedation and/or respiratory arrest), and for mitigating against illegal drug diversion, such as opioid analgesic diversion and/or benzodiazepine diversion.

These pharmaceutical dosage units may be delivered by a variety of delivery methods and/or delivery systems, comprising one or more of: mucosal, sublingual, buccal, nasal inhalation, depot, implantable rod, transdermal patch, parenteral, intravenous (IV), intrathecal, subcutaneous (SC), intramuscular (IM), and the like of delivery means.

Before discussing the various embodiments of the present invention, opioid analgesics, buprenorphine (an example of an opioid analgesic), and benzodiazepine blockers such as flumazenil are discussed.

Opioid analgesics (also referred to herein as narcotic analgesics), such as buprenorphine, are often used when pain control with non-narcotics are ineffective. While narcotic analgesics vary considerably in their chemical structures and pharmacological properties, almost all suffer the disadvantages of tolerance (i.e., loss of efficacy over time) and possible addiction with continued usage.

Narcotic analgesics are classified generally as narcotic agonists or narcotic antagonists. Drugs that activate receptors are termed agonists. Hence, a drug that activates an opioid receptor is termed an opioid agonist. The repeated administration of opioid agonists results in dose-dependent physical dependence and tolerance. Physical dependence manifests as a characteristic set of withdrawal signs and symptoms upon reduction, cessation, or loss of an active compound at an opioid receptor. These opiate withdrawal signs and symptoms may include one or more of the following group comprising: sweating, muscle cramps, physical restlessness, muscle jerks, pupil dilation, bone and/or joint aches, runny nose (rhinorrhea) and/or tearing (lacrimation), nausea, vomiting and/or diarrhea, tremors or shaking, recurrent yawning, anxiety or irritability, goose flesh (hair piloerection), and the like.

A drug that binds to a receptor in a brain neuron to block the receptor rather than activate it is termed an antagonist. Examples of opioid antagonists are naltrexone and naloxone.

Partial agonists are drugs that activate receptors in a brain neuron but not to the extent as full agonists. Buprenorphine is an example of a partial agonist. It is the partial agonist properties of buprenorphine that may contribute to its effectiveness in pain management and provide the added benefit of reduced dependence on and/or reduced addiction to opioids. Consistent with its agonist action at opioid receptors, however, partial agonists such as buprenorphine are still abusable, particularly by individuals who are not already physically dependents on opioids. Note, in some embodiments, a presence of a partial opiate agonist, such as buprenorphine, in a pharmaceutical dosage unit may reduce drug tolerance in that subject, even if full agonist opiates are taken concurrently. Note, in some embodiments, a presence of a partial opiate agonist, such as buprenorphine, in combination with a full opiate antagonist, e.g., naloxone, in the pharmaceutical dosage unit may reduce drug tolerance.

Further, significant data shows that large populations of opiate addicts also abuse benzodiazepines (e.g., with some data showing more than 50 percent of opiate addicts also abusing benzodiazepines). By adding a benzodiazepine blocker to the buprenorphine formulation, or other opioid analgesic formulation, the resulting combination may be far less attractive to opiate addicts. However, the resulting combination may not negatively affect the opioid analgesic and/or may not negatively affect the anti-addictive properties of buprenorphine or other opioid analgesic used in such a combination formulation.

Although buprenorphine's usefulness to effectively manage and treat pain (e.g., chronic and/or sub-acute) and/or to manage and treat opiate addiction may be limited by its diversion potential and the dangers of its co-administration with benzodiazepines, it has now been found that the pharmaceutical dosage unit (e.g., in sublingual administration) comprising a benzodiazepine blocker in combination with relatively high effective amounts of an opioid partial agonist such as buprenorphine, may improve upon existing pain medications and may provide the additional benefit of lessened diversion risk, lessened chance of overdose and reduced side effects.

Opioid analgesic may include at least the following: alfentanil, allylprodine, alphaprodine, anileridine, benzylmorphine, bezitramide, bromadol, buprenorphine, butorphanol, carfentanil, clonitazene, codeine, cyclazocine, desomorphine, dextromoramide, dextropropoxyphene, dezocine, diampromide, diamorphine (heroin), dihydrocodeine, dihydroetorphine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, fentanyl, heroin, hydrocodone, hydromorphone, hydroxypethidine, isomethadone, ketobemidone, levallorphan, levorphanol, levophenacyl morphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papavereturn, pentazocine, pethidine (meperidine hydrochloride), phenadoxone, phenomorphan, phenazocine, phenoperidine, piminodine, piritramide, propheptazine, promedol, properidine, propiram, propoxyphene, sufentanil, tapentadol, tilidine, tramadol, salts thereof, acids thereof, and the like.

Buprenorphine may be a chemical species classified as an opioid, and more specifically as a partial opiate agonist, which may be styled as a mixed agonist-antagonist opioid receptor modulator. Buprenorphine may be a chemical derivative of Thebaine (paramorphine), an alkaloid of the opium poppy, Papaver somniferum. Buprenorphine may be synthesized from Thebaine (paramorphine). As a chemical compound, buprenorphine may not be naturally occurring. Buprenorphine may be legally classified as a narcotic. For example, in the US buprenorphine may be a Schedule III drug, in the United Kingdom (UK) a Class C drug, and in Australia (AU) a Controlled (S8) drug.

Buprenorphine may have a formal IUPAC chemical name of: (25)-2-[(5R,6R,7R,145)-9α-cyclopropylmethyl-4,5-epoxy-6,14-ethano-3-hydroxy-6-methoxymorphinan-7-yl]-3,3-dimethylbutan-2-ol. Buprenorphine may also have a chemical name of: 17-(cyclopropylmethyl)-α-(1,1-dimethylethyl)-4,5-epoxy-18,19-dihydr-o-3-hydroxy-6-methoxy-α-methyl-6,14-ethenomorphian-7-methanol. Buprenorphine may have an atomic weight of 467.64 g/mol. Buprenorphine HCl may have a molecular formula of C₂₉H₄₁NO₄ HCl. Buprenorphine may be a white powder that may be weakly acidic with limited solubility in water. Buprenorphine may have a serum half-life that may vary in humans with 37 hours being an average, with a wide range, often from 20 to 70 hours; which may be sufficiently long to base dosing of off buprenorphine measurements of in blood serum to reach the target serum concentration of buprenorphine of 0.1 ng/mL to 11 ng/mL.

The leading form of buprenorphine is a sublingual film with roughly 60% US market share followed by generic sublingual tablets, and then branded generic tablets. Buprenorphine sublingual tablets may be available from multiple manufacturers. Buprenorphine may be available commercially for parenteral administration under the brand name BUPRENEX (buprenorphine hydrochloride) from Indivior (formerly Reckitt Benckiser, out of Richmond, Va.), and generically as buprenorphine from Abbott Laboratories. Buprenorphine may also be available in sublingual tablets SUBUTEX (buprenorphine HCl) and SUBOXONE (buprenorphine HCl/naloxone HCl dihydrate) both available from Indivior.

SUBOXONE may be available in the US in 8 milligram (mg) and 2 mg tablets and film versions. Buprenorphine may be mixed with Naloxone to prevent IV administration. SUBOXONE's only indication in the US is for opiate addiction and opiate dependence. SUBOXONE is heavily diverted, misused and abused throughout the world.

Buprenorphine may also be available as brand name TEMGESIC SL, a tablet and may be sold at a dosage of 0.3 mg per tablet for pain treatment. TEMGESIC has been available since the 1980's and is not approved nor sold in the US for any indication.

Buprenorphine may have poor oral and/or gastrointestinal bioavailability. Thus buprenorphine is often administered to patients by injection, via a sublingual tablet or film, or as a transdermal patch. Abuse of buprenorphine has been reported to occur via the sublingual and intranasal routes but primarily via diversion of sublingual tablets to the injection route.

Typical analgesic dosage of buprenorphine may be relatively low at amounts of 0.3 to 0.6 mg when injected (intramuscular (IM) or intravenous (IV)). Because the bioavailability of buprenorphine may be less when administered orally or sublingually as compared to injections of buprenorphine, sublingual tablets are available in increased dosages of 2 mg or 8 mg. Indivior may provide a 12 mg sublingual film of buprenorphine

Various embodiments of the present invention, however, may include a relatively high amount (high effective amount) of an opioid analgesic, such as buprenorphine, in a pharmaceutical dosage compared to dosages typically administered for pain management. In one embodiment, a relatively high amount may comprise dosage amounts greater than 0.6 mg of buprenorphine when injected or greater than 8 mg of buprenorphine when administered as a sublingual tablet or film. In one embodiment of the present invention, an opioid analgesic, such as buprenorphine, may be present in an amount of about 2 to 30 mg. In another embodiment, the opioid analgesic may be present in an amount of about 10 to 30 mg per pharmaceutical dosage unit. In still another embodiment, the opioid analgesic may be present in an amount of about 10 to 15 mg per pharmaceutical dosage unit.

As a means to minimize opiate diversion and to generally prevent opiate concurrent use with benzodiazepines, and to reduce a likelihood of overdose (e.g., evidenced by unintended over sedation and/or respiratory arrest) associated with concomitant use of opiates with benzodiazepines, various exemplary embodiments of the present invention may comprise a benzodiazepine blocker, such as flumazenil, in the pharmaceutical dosage unit present along with the one or more opioid analgesics. In some exemplary embodiments, a presence of the flumazenil in the pharmaceutical dosage unit may reduce risks associated with the patient having taken a combination of at least one opiate with at least one benzodiazepine. Such risks may be selected from one or more of the following group consisting of: unintended over sedation, respiratory arrest, death, and the like.

Flumazenil as a benzodiazepine blocker may be a benzodiazepine receptor antagonist. Flumazenil may be the only benzodiazepine receptor antagonist (i.e., benzodiazepine blocker) on the market today. Flumazenil currently is not a narcotic nor a controlled substance in the US Flumazenil may have been first introduced in 1987 by Hoffmann-La Roche under the trade name ANEXATE, which may have been approved by the FDA on Dec. 20, 1991. Flumazenil may also be available under the trade names of LANEXAT, MAZICON, and ROMAZICON.

Flumazenil may also be known as flumazepil. Flumazenil may have a code name of: Ro 15-1788. Flumazenil may have a formal IUPAC chemical name of: ethyl 8-fluoro-5-methyl-6-oxo-5,6-dihydro-4H-benzo[f]imidazo[1,5-a][1,4]diazepine-3-carboxylate. Flumazenil may have a chemical formula of C15H14FN3O3 and an atomic weight of 303.288 g/mol (i.e., 303.8 Daltons). Flumazenil may have an elimination half-life of 84 minutes. Such a half-life is considerably shorter than as compared to buprenorphine; and thus flumazenil dosing may not lend itself to dosing based of measuring flumazenil in blood serum. Instead, in some embodiments, flumazenil dosing may be based on visible patient effect.

Flumazenil may have traditionally been available in liquid form for injection and IV use as flumazenil may have a very low oral absorption (bioavailability). In various exemplary embodiments of the present invention, flumazenil (and the one or more opioid analgesics) may be administered sublingually or buccally thereby bypassing a first pass liver removal and metabolism and markedly improving adsorption (bioavailability).

Now turning to specific formulations of the pharmaceutical dosage unit. In some embodiments, the pharmaceutical dosage unit may comprise an opioid analgesic and a benzodiazepine blocker. In such embodiments, wherein the pharmaceutical dosage unit comprises at least one opioid analgesic and the benzodiazepine blocker, those two different chemical species may be both present in the same the pharmaceutical dosage unit. In some embodiments, those two different chemical species may be both present in the same the pharmaceutical dosage unit in a manner wherein those different chemical species may be substantially randomly and substantially evenly mixed (distributed) within the pharmaceutical dosage unit, such that separating one chemical species from the other may be mechanically difficult.

In some embodiments, the pharmaceutical dosage unit may comprise at least one (or one or more) opioid analgesic present in a first therapeutically effective amount and a benzodiazepine blocker present in a second therapeutically effective amount. In some embodiments, the buprenorphine or the at least one opioid analgesic may be present in the first therapeutically effective amount and the flumazenil may be present in the second therapeutically effective amount. The first and the second therapeutically effective amounts may vary according to a delivery method of administration. For example, and without limiting the scope of the present invention, the first therapeutically effective amount may be an amount that results in a target serum concentration of buprenorphine of 0.1 ng/mL to 11 ng/mL.

In some embodiments, the opioid analgesic may be present in a range of about 1 to 61 mg. In some embodiments, the opioid analgesic may be present in a range of about 2 to 24 milligrams. In some embodiments, the opioid analgesic may present in an amount of about 4 mg to about 8 mg. In some embodiments, the benzodiazepine blocker may be present in a range of about 0.15 to 61 mg. In some embodiments, the benzodiazepine blocker may be present in a range of about 2 to 14 mg.

In some embodiments, the opioid analgesic may be buprenorphine and the benzodiazepine blocker may be flumazenil. In some embodiments, the flumazenil may be present in a range of about 0.15 to 61 mg or in a range of about 2 to 60 mg or in a range of about 0.25 to 60 mg. In some embodiments, the flumazenil may be present in the pharmaceutical dosage unit at about 2 to 4 mg, wherein such a pharmaceutical dosage unit may be administered multiple times daily to the patient (e.g., from two to seven times daily). In some embodiments, the flumazenil may be present in the pharmaceutical dosage unit at about 4 to 14 mg, wherein such a pharmaceutical dosage unit may be administered at least once daily to the patient. Such a presence of flumazenil in the pharmaceutical does unit may limit or prevent concomitant usage of benzodiazepines with opioid analgesics. Since benzodiazepine addiction and dependence may be rampant and growing steadily among the opiate addiction community, the addition of flumazenil may markedly improve buprenorphine's and/or other opioid analgesics safety (e.g., reducing and/or minimizing overdoses) and decrease opiate diversion and black market sales.

The amounts of flumazenil present in the various embodiments of the pharmaceutical dosage unit (e.g. 0.15 to 61 mg) may be for preventing and/or reversing concurrent use of benzodiazepines with opiates. Flumazenil may be used in patients using opiate receptor based drugs to treat pain and/or addiction and the benzodiazepine blocker (flumazenil) may be administered within the delivery means (e.g., sublingual tablet) at the same time and effectively prevent the accidental combination overdose. Because the various embodiments of the pharmaceutical dosage unit may comprise flumazenil (a benzodiazepine blocker), the pharmaceutical dosage unit may be far less attractive for diversion or for sale in the illegal street drug market.

Note, several historical physiological ideas about IV flumazenil may be much different compared to the formulations of flumazenil used in the various pharmaceutical dosage units. For example, tablet pilot studies may have shown flumazenil does not cause anxiety nor panic attacks and there may be no dysphoria nor disorientation as historically noted in IV flumazenil. When patients are currently prescribed opiates, such patients are instructed to avoid benzodiazepines and the pharmaceutical dosage units disclosed herein may make physicians more confident that such patients will remain compliant with that instruction.

Additionally, note, phenobarbital, TEGRETOL (carbamazepine), and DEPAKOTE (valproic acid) are not benzodiazepines and may be safe to take in patients also taking flumazenil. Thus in some embodiments, patients may concomitantly use phenobarbital, TEGRETOL, or DEPAKOTE with the various embodiments of the pharmaceutical dosage unit without a significant reduction in the efficacy of the pharmaceutical dosage unit.

In some embodiments, the buprenorphine may be present in a range of about 6 to 10 mg and the flumazenil may be present at about 2 mg. In some embodiments, such a formulation of the pharmaceutical dosage unit may be administered several times daily for treating pain (e.g. chronic pain) and/or addiction. Several times daily may from one to seven treatments per day. In some embodiments, a frequency of multiple dosing per day (e.g. two to seven) may be more important than a mg variance (e.g., 6 to 10 mg) in the buprenorphine because of how buprenorphine may be metabolized in the body. For example, and without limiting the scope of the present invention, the multiple times per day dosing of buprenorphine may seek to result in the target serum concentration of buprenorphine of 0.1 ng/mL to 11 ng/mL.

In some embodiments, the buprenorphine may be present in an amount of about 4 mg. And the flumazenil may be present in an amount of about 2 mg, 2.5 mg, or 4.5 mg; or the flumazenil may be present in about 2 mg to about 5 mg.

The 4 mg buprenorphine with 2 mg flumazenil may be useful for treating chronic pain, sub-acute pain, and/or for opiate addiction treatment. For such treatments, this 4 mg buprenorphine with 2 mg flumazenil embodiment may be administered up to four times daily to the patient.

The 4 mg buprenorphine with 2.5 mg flumazenil may be useful for treating opiate addiction, particularly that of hydrodone and/or oxycontin addiction. For such addiction treatment, this 4 mg buprenorphine with 2.5 mg flumazenil formulation embodiment may be administered three times daily to the addiction patient. See e.g., a discussion regarding Example 1 below regarding this 4 mg buprenorphine with 2.5 mg flumazenil formulation embodiment.

The 4 mg buprenorphine with 4.5 mg flumazenil may be useful for treating chronic pain, sub-acute pain, and/or for opiate addiction treatment. For such treatment, this 4 mg buprenorphine with 4.5 mg flumazenil embodiment may be administered twice times daily to the patient.

In some embodiments, the buprenorphine may be present in a range of 12 to 30 milligrams and the flumazenil may be present in a range of about 2 to 15 mg. In some embodiments, such as formulation of the pharmaceutical dosage unit may be administered once daily for treating chronic pain, sub-acute pain, and/or opiate addiction, depending upon which partial opiate agonist and/or full opiate agonist may be delivered with the flumazenil.

In some embodiments, the buprenorphine may be present in an amount of about 2, 4, or 8 mg; or the buprenorphine may be present in an amount of about 2 mg to about 8 mg. And the Flumazenil may be present in an amount of about 2 mg.

The 2 mg buprenorphine with 2 mg flumazenil may be useful for treating opiate addiction, such as, but not limited to, methadone addiction. For such opiate addiction treatment, this 2 mg buprenorphine with 2 mg flumazenil embodiment may be administered up to four times daily to the patient. See e.g., a discussion regarding Example 2 below regarding this 2 mg buprenorphine with 2 mg flumazenil formulation embodiment.

The 4 mg buprenorphine with 2 mg flumazenil may be useful for treating opiate addiction, chronic pain, and/or sub-acute pain. For such treatment, this 4 mg buprenorphine with 2 mg flumazenil embodiment may be administered twice times daily to the patient.

The 8 mg buprenorphine with 2 mg flumazenil may be useful for treating opiate addiction, chronic pain, and/or sub-acute pain. For such treatment, this 8 mg buprenorphine with 2 mg flumazenil embodiment may be administered twice times daily to the patient.

In some embodiments, the buprenorphine may be present in an amount of about 2, 4, or 8 mg; or the buprenorphine may be present in an amount of about 2 mg to about 8 mg. And the Flumazenil may be present in an amount of about 2.5 mg.

In some embodiments, the pharmaceutical dosage unit may comprise at least one opioid antagonist. In some embodiments, the at least one opioid antagonist may be naloxone. In some embodiments, the naloxone may be present in about 0.2 mg per pharmaceutical dosage unit. In embodiments with such an inclusion of naloxone, presence of naloxone in the pharmaceutical dosage unit may reduce opiate tolerance or even partially reverse tolerance if opiate tolerances occurs. Naloxone may be included in the pharmaceutical dosage unit to further reduce the abuse potential of the pharmaceutical dosage unit and withdrawal as a result of physical dependence and/or addiction.

In some embodiments, the opioid analgesic (of the pharmaceutical dosage unit) may be selected from one or more of the group comprising: alfentanil, allylprodine, alphaprodine, anileridine, benzylmorphine, bezitramide, bromadol, buprenorphine, butorphanol, carfentanil, clonitazene, codeine, cyclazocine, desomorphine, dextromoramide, dextropropoxyphene, dezocine, diampromide, diamorphine (heroin), dihydrocodeine, dihydroetorphine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, fentanyl, heroin, hydrocodone, hydromorphone, hydroxypethidine, isomethadone, ketobemidone, levallorphan, levorphanol, levophenacyl morphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papavereturn, pentazocine, pethidine (meperidine hydrochloride), phenadoxone, phenomorphan, phenazocine, phenoperidine, piminodine, piritramide, propheptazine, promedol, properidine, propiram, propoxyphene, sufentanil, tapentadol, tilidine, tramadol, salts thereof, acids thereof, and the like.

In some embodiments, the opioid analgesic may be hydromorphone and the benzodiazepine blocker may be flumazenil (e.g., with the flumazenil at 2 mg). In some embodiments, the hydromorphone may be present in an amount of about ½, 1, or 2 mg (e.g., per tablet); or the hydromorphone may be present in an amount of about ½ mg to about 2 mg per tablet. In some embodiments, the hydromorphone may be present in an amount of about ½ mg to 9 mg (e.g., per tablet). And the flumazenil may be present in an amount of about 2 mg per tablet. In some embodiments, such a formulation of the pharmaceutical dosage unit may be administered up to three times daily for treating pain (chronic and/or sub-acute). In some embodiments, such a formulation of the pharmaceutical dosage unit may be administered up to four times daily for treating pain (chronic and/or sub-acute).

In some embodiments, the opioid analgesic may be hydrocodone and the benzodiazepine blocker may be flumazenil. In some embodiments, the hydrocodone may be present in an amount of about 10, 12.5, or 15 mg (e.g., per tablet). In some embodiments, the hydrocodone may be present in an amount of about 2.5 mg to 31 mg (e.g., per tablet). In some embodiments, the flumazenil may be present in an amount of about 2 mg (e.g., per tablet). In some embodiments, the flumazenil may be present in an amount of about 2 mg to about 24 mg (e.g., per tablet). In some embodiments, such as formulation of the pharmaceutical dosage unit may be administered up to four times daily for treating pain (chronic and/or sub-acute).

In some embodiments, the opioid analgesic may be oxycodone and the benzodiazepine blocker may be flumazenil. In some embodiments, the oxycodone may be present in an amount of about 10, 15, or 20 mg (e.g., per tablet). In some embodiments, the oxycodone may be present in an amount of about 2.5 to 31 mg (e.g., per tablet). In some embodiments, the flumazenil may be present in an amount of about 2 mg (e.g., per tablet). In some embodiments, the flumazenil may be present in an amount of about 2 mg to about 24 mg (e.g., per tablet). In some embodiments, such as formulation of the pharmaceutical dosage unit may be administered up to four times daily for treating pain (chronic and/or sub-acute).

In some embodiments, the opioid analgesic may be morphine and the benzodiazepine blocker may be flumazenil. In some embodiments, the morphine may be present in an amount of about 10, 20, or 30 mg (e.g., per tablet). In some embodiments, the morphine may be present in an amount of about 9 to 41 mg (e.g., per tablet). In some embodiments, the flumazenil may be present in an amount of about 2 mg (e.g., per tablet). In some embodiments, the flumazenil may be present in an amount of about 2 mg to about 24 mg (e.g., per tablet). In some embodiments, such as formulation of the pharmaceutical dosage unit may be administered up to four times daily for treating pain (chronic and/or sub-acute).

In some embodiments, the opioid analgesic may be methadone and the benzodiazepine blocker may be flumazenil. In some embodiments, the methadone may be present in an amount of about 20 mg per milliliter (mL) of the pharmaceutical dosage unit. In some embodiments, the methadone may be present in an amount of about 10 mg per milliliter (mL) of the pharmaceutical dosage unit. And the flumazenil may be present in an amount of about ¼ mg per mL to about 1 mg per mL of the pharmaceutical dosage unit. For example, and without limiting the scope of the present invention, in some embodiments, the flumazenil may be present in an amount of about ¼ mg per mL, or about ½ mg per mL, or about 1 mg per mL of the pharmaceutical dosage unit. In some embodiments, flumazenil may be present in an amount up to and including 10 mg per mL of the pharmaceutical dosage unit. In some embodiments, about 5 to about 300 mg of methadone in liquid format may be delivered once daily for treating pain (chronic and/or sub-acute) and addiction. Although while useful for treating pain and/or addiction, presently in the US methadone may be dispensed for addiction treatment only at licensed addiction clinics, but could be prescribed for addiction in clinical settings.

In some embodiments, the opioid analgesic may be codeine and the benzodiazepine blocker may be flumazenil. In some embodiments, the codeine may be present in an amount of about 15, 30 or 60 mg (e.g., per tablet). In some embodiments, the codeine may be present in an amount of about 14 to 61 mg (e.g., per tablet). In some embodiments, the flumazenil may be present in an amount of about 2 mg (e.g., per tablet). In some embodiments, the flumazenil may be present in an amount of about 2 mg to about 24 mg (e.g., per tablet). In some embodiments, such as formulation of the pharmaceutical dosage unit may be administered up to four times daily for treating pain (chronic and/or sub-acute).

In some embodiments, the pharmaceutical dosage unit may be administered to the patient in one or more of a variety of delivery methods. Such delivery methods may be selected from one or more of the group comprising: mucosal, sublingual (e.g., tablet and/or film), buccal, nasal inhalation, depot, implantable rod, transdermal patch, parenteral, intravenous (IV), intrathecal, subcutaneous (SC), intramuscular (IM), and the like.

To the extent any given embodiment of the pharmaceutical dosage unit comprises buprenorphine, buprenorphine dosing (presence) in any of the above delivery methods may be arrived at by measuring blood serum for buprenorphine; and when dosing provides the target serum concentration of buprenorphine of 0.1 ng/mL to 11 ng/mL such dosing of buprenorphine in the pharmaceutical dosage unit may then be appropriate.

Consistent with these various delivery methods of administration, in some embodiments, the benzodiazepine blocker (e.g., flumazenil) and the one or more opioid analgesics may be combined in the same formulation (compounding) of the pharmaceutical dosage unit, such that at least some of the benzodiazepine blocker (e.g., flumazenil) and the one or more opioid analgesics may be in physical contact with each other. In some embodiments, the benzodiazepine blocker (e.g., flumazenil) and the one or more opioid analgesic may be provided as individual (separate and distinct) formulations but administered such that the benzodiazepine blocker (e.g., flumazenil) and the one or more opioid analgesic may be delivered to the patient simultaneously (e.g., a double barrel syringe delivery system).

Mucosal drug delivery may be an alternative method of systemic drug delivery that may offer some advantages over both injectable and enteral (gastrointestinal) methods. Mucosal drug delivery may generally refer to bringing the pharmaceutical dosage unit within physical contact with one or more mucosal tissues, which are generally heavily vascularized, such as oral mucosa, nasal mucosa, and/or rectal mucosa. The oral mucosa may comprise sublingual regions below the tongue and buccal regions of the inner cheeks.

Note, where specific sublingual formulations may be disclosed herein, such embodiments may be reformulated for buccal delivery in a comparatively similar formulation that is still within the scope of this invention. Likewise, where specific buccal formulations may be disclosed, such embodiments may be reformulated for sublingual delivery in a comparatively similar formulation that is still within the scope of this invention. That is, sublingual and buccal formulations may be substantially equivalent.

In some embodiments, the pharmaceutical dosage unit may be formulated so that the pharmaceutically active components (e.g., the one or more opioid analgesic and the flumazenil, and/or the naloxone) may exhibit sustained-release characteristics upon administration to the patient. For example, the pharmaceutically active components may be delivered with an oral mucosal patch. For example, and without limiting the scope of the present invention, the dosing of buprenorphine may result in the target serum concentration of buprenorphine of 0.1 ng/mL to 11 ng/mL. Methods of making such patches are well known to one of ordinary skill in the art. In some embodiments the oral mucosal patch may be prepared by homogeneously mixing buprenorphine (or other opioid analgesic) and flumazenil with appropriate amounts of Carbopol 934, polyisobutylene, and polyisoprene using a two-roll mill and then compressing the mixture to the appropriate thickness. A membrane backing such as ethylcellulose may then applied to one side of the compressed material and circular disks, having an area of about 0.5 cm² and thickness of about 0.6 mm for example, may be punched from the material. The backing may inhibit drug release from one side of the disk and may reduce adhesion to opposing side tissues. Such oral mucosal patches may be secured to mucosal buccal surfaces such as the gums, lips, and cheeks, and worn for extended periods. In some embodiments, such an oral mucosal patch may be work for about 12 hours.

Note, as used in this disclosure, the phrase “sustained-release” may indicate that an pharmaceutically active component may be released from the pharmaceutical dosage unit over a period of time which may be longer than the pharmaceutically active component's ordinary in vivo half-life, thus extending the presence of the pharmaceutically active component in a patient's system considerably beyond its ordinary half-life. Under such circumstances, the pharmaceutically active components may be said to “exhibit sustained-release characteristics.” In contrast, the phrase “immediate-release” may indicate that an pharmaceutically active component may be released from the pharmaceutical dosage unit within a comparatively shorter period of time (i.e., shorter than its ordinary in vivo half-life); and decrease in concentration of the pharmaceutically active components over time may be approximately described by its ordinary half-life. Under such circumstances, the pharmaceutically active components may be said to “exhibit immediate-release characteristics.” In compositions and/or formulations of various embodiments having more than one pharmaceutically active component that may be intended to exhibit sustained-release-characteristics, an important consideration may be that the release rate of each pharmaceutically active components may be separately customized to produce the desired release profile, since the pharmaceutically active components may have different in vivo half-lives.

In some exemplary embodiments, the various pharmaceutical dosage units may be in a sublingual tablet and/or sublingual film. Sublingual delivery may refer to a pharmacological route of administration (delivery) by which drugs may diffuse into the blood through tissues (e.g., sublingual mucosa) under the tongue. Such a route may avoid first-pass metabolism and may afford quick drug entry into the systemic circulation of the blood. Sublingual delivery may be in tablet (pill) formulation, capsule formulation and/or film formulation.

Sublingual tablets may be designed to dissolve very rapidly once in the oral cavity. Necessary ingredients for the pharmaceutical dosage unit may be processed in accordance with known methods, using or incorporating familiar coatings and additives as required.

In some embodiments, the pharmaceutical dosage unit may be a sublingual tablet. In some embodiments, the sublingual table may be comprise buprenorphine as the opioid analgesic. In some embodiments, the buprenorphine may be present in an amount of about 4 mg per sublingual tablet. The sublingual table may be comprise flumazenil as the benzodiazepine blocker. The flumazenil may be present in an amount of about of 2 mg per sublingual tablet.

By way of example only and without limiting the scope of the present invention, in addition to the pharmaceutically active components (e.g., the opioid analgesic and benzodiazepine blocker), the pharmaceutical dosage unit may comprise effective amounts of one or more of: binders, fillers, disintegrants, sustained-release agents, diluents, anti-adherents, glidants, flow aids, plasticizers and lubricants, which are well known in the field of pharmaceutical processing. Pharmaceutically active components may also be known as “active ingredients.” For instance, the formulation of these sublingual tablets may comprise, in addition to the pharmaceutically active components, a limited number of soluble excipients, including a binder such as povidone or hydroxypropyl methylcellulose (HPMC), diluents such as lactose, mannitol, starch or cellulose, a disintegrant such as pregelatinized or modified starch, lubricants such as magnesium stearate, stearic acid or hydrogenated vegetable oil, a sweetener such as saccharin and/or sucrose and suitable flavoring and coloring agents. The process of making sublingual tablets may generally involve moistening the blended powder components with an alcohol-water solvent system containing approximately about 60 percent alcohol and about 40 percent water and pressing this mixture into tablets. Such formulation and/or compounding techniques may be well known in the art to one of ordinary skill in the art.

For example, and without limiting the scope of the present invention, in some embodiments, the above 4 mg buprenorphine and 2 mg flumazenil sublingual tablets may also comprise Sugar Base A. In other embodiments, other sweeteners and/or sugar bases may be used.

For example, and without limiting the scope of the present invention, in some embodiments, the Sugar Base A may comprise: DiPac at about 77 percent by weight by per tablet, lactose at about 7 percent by weight by per tablet, microcrystalline cellulose at about 5 percent by weight by per tablet, flavoring at about 5 percent by weight by per tablet, crospovidone at about 5 percent by weight by per tablet, and magnesium stearate at about 1 percent by weight per tablet. DiPac may be a sucrose or sugar based diluents. In other embodiments, other sucrose or sugar based diluents may be used in place of DiPac. Crospovidone may be an excipient in pharmaceutical compounding. Crospovidone may be used as a solubility enhancer, adsorbent, coating material, carrier, disintegrant, extrusion spheronization aid, and recrystallization inhibitor.

For example, and without limiting the scope of the present invention, in some embodiments, the above 4 mg buprenorphine and 2 mg flumazenil sublingual tablets may also comprise Coloring A. Coloring A may be added in about 20 mg of Coloring A per 150 sublingual tablets when the sublingual tablet formulation may be being prepared. In other embodiments, other colorant formulations may be used.

In some embodiments, the above 4 mg buprenorphine and 2 mg flumazenil sublingual tablet may also comprise a third pharmaceutically active component, such as an opioid antagonist. In some exemplary embodiments the opioid antagonist may comprise naloxone. In some embodiments, the naloxone may be present in about 0.2 mg per sublingual tablet to about 1 mg per sublingual tablet. In embodiments, presence of naloxone in the pharmaceutical dosage unit may reduce opiate tolerance or even partially reverse tolerance if opiate tolerances occurs. Naloxone may be included in the pharmaceutical dosage unit to further reduce the abuse potential of the pharmaceutical dosage unit and withdrawal as a result of physical dependence and/or addiction. Such a 4 mg buprenorphine, 2 mg flumazenil, and 0.2 mg naloxone sublingual tablet may comprise Sugar Base A and/or Coloring A per above.

With respect to these sublingual tablets, such sublingual tablets may be placed within the mouth and/or under the tongue. In some embodiments, the pharmaceutical dosage unit may be presented in a single dose or as divided doses administered at appropriate intervals, for example, as two, three, or four sub-doses per day. In some embodiments, the pharmaceutical dosage unit may be presented in divided doses administered at appropriate intervals, for example, as two, three, four, five, six, seven, or eight sub-doses per day. In one embodiment, the pharmaceutical dosage unit may be formulated such that a single sublingual tablet may be administered twice daily. For example and without limiting the scope of the present invention, the pharmaceutical dosage unit may comprise about 4 mg buprenorphine and about 2 to 4.5 mg of flumazenil. In one embodiment, pharmaceutical dosage unit of the present invention, e.g. in sublingual tablet format, may be delivered once in the morning, such as at around 8:00 am, and then again about six hours later in the early afternoon or at approximately 2:00 pm.

Buccal administration (delivery) may refer to a topical route of administration by which drugs may partially diffuse through the oral mucosa (e.g., tissues which may line the mouth) and may then enter directly into the bloodstream. Buccal administration typically result in higher bioavailability of a drug a more rapid onset of action as compared against digestive tract administration. This is because the medication does not pass through the digestive system and thereby avoids first pass metabolism.

In another sustained-release embodiment, the pharmaceutically active components may be delivered using a tablet-form pharmaceutical dosage unit having a partially hydrophilic matrix which may exhibit sustained-release of at least one of the pharmaceutically active components (e.g. buprenorphine or flumazenil). In addition to the pharmaceutically active components, the a tablet may be comprised of, for example, ethylcellulose as a sustained-release agent and hydroxypropyl methylcellulose (HPMC) as a film former. Further, bulking agents such as microcrystalline cellulose and starch, a polyvinylpyrrolidone binder, silicon dioxide as an anti-adherent, dibutyl sebacate as a plasticizer, and magnesium stearate as a lubricant may be included. Using conventional processes, the listed ingredients, other than ethylcellulose, HPMC and dibutyl sebacate, may be combined and pressed into a tablet. The tablet may be then coated with the ethylcellulose, HPMC and/or dibutyl sebacate prior to administration of the tablet. When this tablet encounters an aqueous environment, such as mucosal buccal and/or sublingual surfaces, portions of the tablet coating may dissolve, leaving a non-continuous film of water-insoluble ethylcellulose surrounding the remaining tablet core. The rate of diffusion of the pharmaceutically active components from the tablet core into the aqueous environment may be determined by the concentration of ethylcellulose, HPMC and/or dibutyl sebacate in the coating.

Nasal inhalation delivery may refer refers to a pharmacological route of administration by which drugs may diffuse into the blood through the thin mucosa within the nasal cavity (e.g., the single epithelial cell layer lining some of the nasal cavity). Such a route may avoid first-pass metabolism and may afford quick drug entry into the systemic circulation of the blood. Pharmaceutical dosage unit formulations for nasal inhalation delivery may be powder and/or liquid formulations.

Depot delivery may refer to a pharmacological route of administration by which drugs may be injected (or surgically implanted) into the body, generally through a subcutaneous (SC) or intramuscular (IM) injection. However, in depot delivery systems, the drugs may be formulated for controlled release, extended release, and/or sustained-release. Such formulations may be achieved by oil based formulations, suspension based formulations, and/or polymer based microspheres or rods. Depot delivery may offer an advantage of a very high loading, controlled release of drug for an extended period of time and may reduce frequency of dosing.

For example, implantable rods may be used as a drug reservoir, wherein the drug formulation may be released in a controlled and near constant manner providing for a sustained-release dosing regimen. Such implantable rods may be substantially constructed of various biomaterials, i.e. materials which may generally be considered to be nonviable materials, which may be chosen to minimize immune responses. For example, and without limiting the scope of the present invention, such biomaterials may be various plastic based polymers, other polymers (e.g. biodegradable collagen), non-biodegradable titanium metal, and the like.

A transdermal patch may be a medicated adhesive patch that may be placed on the skin to deliver a specific dose of medication through the skin and into the bloodstream. At least one advantage of a transdermal drug delivery route over other types of drug delivery such as oral, topical, intravenous, intramuscular may be that such patch provide a controlled release of the medication into the patient, usually through either a porous membrane covering a reservoir of medication or through body heat melting thin layers of medication embedded in the adhesive. However, at least one traditional disadvantage to many transdermal delivery systems may be that skin may be an effective barrier against entry for larger molecules. For example, the stratum corneum layer in the skin may provide a strong barrier against entry of molecules larger than 500 Dalton. As a result, only medications whose molecules are small enough to penetrate the skin can be delivered by this method. For example, and without limiting the scope of the present invention, pharmaceutical dosage units comprising the one or more opioid analgesic (e.g., buprenorphine) and flumazenil may be delivered by transdermal patch, as the molecular size of such molecules generally falls within the molecule size for successful transdermal drug delivery.

Parenteral drug delivery methods and systems may comprise delivering a pharmaceutical dosage unit by injection, infusion, and/or implantation. Thus, parenteral delivery means may comprise intravenous (IV), intrathecal, subcutaneous (SC), intramuscular (IM), and other parenteral routes. IV injection may be direct delivery to the veins resulting in quick systemic delivery. IV injection may comprise use of an injection directly to a vein, or delivery of medication to a catheter, wherein the catheter may be inserted into a vein. The IV route may also utilize an IV bolus for delivery, an IV drip (IV infusion), and/or use of various metering pumps.

Intrathecal drug delivery may comprise use of a very controlled pump to deliver medication (e.g., an analgesic) directly to a region of the spinal cord. The pump may be surgically placed under the abdominal skin and may deliver the analgesic through a catheter to the region of the spinal cord.

Subcutaneous (SC) delivery may refer to administration of the drug beneath the skin. Generally this may be done by injection below the epidermis and dermis layers of the skin. Subcutaneous delivery may sometimes be referred to as hypodermic injection.

Intramuscular (IM) delivery may refer to administration of the drug to a muscle, generally by injection, e.g., into the muscles of the upper arm or gluteal area.

In some embodiments, the various pharmaceutical dosage units, including the various delivery methods, may be administered in various methods to the patient for one or more purposes of: pain, chronic pain, sub-acute pain, chronic and sub-acute pain, anxiety, addiction in general, opiate addiction, and benzodiazepine addiction, opiate and benzodiazepine addiction; opiate dependence, benzodiazepine dependence; and for reducing a likelihood of overdose associated with concomitant use of opiates with benzodiazepines (e.g., evidenced by unintended over sedation and/or respiratory arrest), and for mitigating against drug diversion, such opioid analgesics and/or benzodiazepines. In some embodiments, treating addiction may also entail treating withdrawal.

Note, all the pharmaceutical dosage unit embodiments, as well as all methods of treatment may comprise the pharmaceutical dosage unit comprising one or more opioid analgesics with the benzodiazepine blocker, such as flumazenil; or may comprise near simultaneous administration to the patient of one or more opioid analgesics along with the benzodiazepine blocker, such as flumazenil—such that all embodiments may mitigate against drug diversion, as well as mitigate against overdose associated with benzodiazepine combined use with an opiate. As the presence of the benzodiazepine blocker (e.g., flumazenil) makes such pharmaceutical dosage unit less ideal for diversion and may increase prescribing physician comfort and/or confidence when prescribing an opioid analgesic for pain treatment and/or for treating opioid addiction.

In some embodiments, a method for treating pain (chronic and/or sub-acute) may comprise a step of administering to the patient the pharmaceutical dosage unit. In some such methods of treatment embodiments, the pharmaceutical dosage unit may comprise from about 1 mg to 61 mg of an opioid analgesic, such as buprenorphine; and may further comprise from about 0.15 mg to 61 mg of flumazenil (or another benzodiazepine blocker). The flumazenil and the opioid analgesic may be administered sublingually as a single pharmaceutical dosage unit or simultaneously in separate, sublingual pharmaceutical dosage units. In some embodiments, the pharmaceutical dosage unit for treating pain may be by administration of a sublingual tablet (or film) to be placed under the tongue or other delivery method as noted above.

In some embodiments, methods for pain treatment may additionally include a step of monitoring the patient after an initial administration of the pharmaceutical dosage unit to determine if the dosage regime may be appropriate and either increasing or decreasing the dosage regimen as necessary. For example, the first therapeutically effective amount in the pharmaceutical dosage unit may be such that results in serum concentration of buprenorphine of 0.1 ng/mL to 11 ng/mL.

Where a specific dosage of administration for the various pharmaceutical dosage units may not be expressly disclosed herein, such appropriate dosages for administration to the patient may be determined in accordance with accepted guidelines, such as those given by the Physician's Desk Reference. The patient's response to the various pharmaceutical dosage units of the present invention may be monitored and the dosage adjusted as necessary.

In some embodiments, methods for treating pain (chronic and/or sub-acute) may result in reduced side effects and/or reduced severity in side effects as compared to existing opioid analgesic methods for treating pain. For example, and without limiting the scope of the present invention, such side effects may comprise opioid withdrawal (opioid addiction).

For example, and without limiting the scope of the present invention, in some embodiments the method for treating pain, which may result in reduced side effects and/or reduced severity in side effects, may comprise the step of administering the pharmaceutical dosage unit to the patient, wherein the pharmaceutical dosage unit may comprise from about 1 mg to 61 mg of the opioid analgesic, such as buprenorphine; and may further comprise from about 2 mg to 5 mg of flumazenil. The opioid analgesic and flumazenil may be administered sublingually as a single pharmaceutical dosage unit or simultaneously in separate, sublingual pharmaceutical dosage units.

In some embodiments, a method for treating opioid addiction (opioid withdrawal) may comprise a step of administering to the patient the pharmaceutical dosage unit. In some such methods of treatment embodiments, the pharmaceutical dosage unit may comprise from about 1 mg to 31 mg of an opioid analgesic, such as buprenorphine; and may further comprise from about 1 mg to 6 mg of flumazenil. The flumazenil and the opioid analgesic may be administered sublingually as a single pharmaceutical dosage unit or simultaneously in separate, sublingual pharmaceutical dosage units. In some embodiments, the pharmaceutical dosage unit for treating opioid addiction (opioid withdrawal) may be by administration of a sublingual tablet (or film) to be placed under the tongue or other delivery method as noted above.

In some embodiments, methods for treating opioid addiction (opioid withdrawal) may additionally include a step of monitoring the patient to determine if the dosage regime may be appropriate and either increasing or decreasing the dosage regimen as necessary.

Further objects and advantages of the invention will become apparent from a consideration of the examples and ensuing description which illustrate embodiments of the invention, it being understood that the foregoing statements of the objects of the invention are intended to generally explain the same without limiting it in any manner.

### EXAMPLE 1

Example 1 may address a method for treating pain (chronic and/or sub-acute), wherein the method may comprise administering the pharmaceutical dosage unit comprising about 4 mg buprenorphine and about 2.5 mg flumazenil in a sublingual tablet, three times daily to patients who had been using hydrocodone or oxycodone for treating the patients' pain. Additionally, inclusion of the flumazenil in the pharmaceutical dosage unit may result in a reduced likelihood of overdose from concomitant use of an opioid analgesic with a benzodiazepine. Additionally, inclusion of the flumazenil in the pharmaceutical dosage unit may result in mitigating against illegal diversion of the pharmaceutical dosage unit.

Patients who were taking 40 mg to 80 mg per day of hydrocodone or 40 mg to 320 mg per day of oxycodone were switched to a pharmaceutical dosage unit comprising about 4 mg buprenorphine and about 2.5 mg flumazenil in a sublingual tablet. The sublingual tablets were administered to each patient three times daily. The patient's perceived pain was measured using a visual analog scale (VAS).

The patients formerly taking 40 mg to 80 mg per day of hydrocodone experienced a 42 percent decrease in perceived pain scores after conversion to the about 4 mg buprenorphine and the about 2.5 mg flumazenil sublingual tablet taken 3 times daily. The perceived pain scores also remained stable when a half dose reduction was attempted after an 84 day stabilization phase.

The patients that had been taking 40 mg to 320 mg per day of oxycodone experienced a 34 percent reduction in perceived pain 96 hours after conversion to the about 4 mg buprenorphine and the about 2.5 mg flumazenil sublingual tablet taken 3 times daily. No withdrawal or parasympathetic symptoms were noted in the patients after this conversion. The perceived pain scores remained stable when a dose reduction was attempted after a 40-day stabilization phase.

The patients, in both the hydrocodone and the oxycodone groups, also generally reported feelings of being more in control of their lives, thinking clearer, sleeping better and being less lethargic than when they were taking hydrocodone or oxycodone.

Thus, in terms of unexpected results, this study utilizing the sublingual tablet pharmaceutical dosage unit of the about 4 mg buprenorphine and the about 2.5 mg flumazenil taken 3 times daily demonstrated enhanced pain relief as well as an unexpected increase in wellbeing; as compared against a pain treatment regime using 40 mg to 80 mg per day of hydrocodone or 40 mg to 320 mg per day of oxycodone.

### EXAMPLE 2

Patients who had been taking 20 mg to140 mg per day of methadone were switched to a pharmaceutical dosage unit comprising about 2 mg buprenorphine together with about 2 mg flumazenil in a sublingual tablet. These patients may have initially been taking methadone for pain and became addicted to methadone. These patients took an average of 9 days to convert before methadone withdrawal was completely eliminated.

Patients who had been taking 20 mg to 50 mg per day of methadone converted without ill effects, but did not take the pharmaceutical dosage unit comprising the about 2 mg buprenorphine and the about 2 mg flumazenil in the sublingual tablet on the first day.

Patients who had been taking 50 mg to 140 mg per day of methadone were first titrated down with Ultram (e.g., via 2 to 3 tablets of Ultram 50 mg administered every 6 hours) for four days. Following this titration period, the patients were given injections containing 0.3 mg buprenorphine and 60 mg torodol to see if further time was needed before the sublingual tablets comprising buprenorphine and flumazenil could be started without risk of precipitating withdrawal. Significant withdrawal was observed in 24 percent of the patients on the first day the about 2 mg buprenorphine and the about 2 mg flumazenil sublingual tablets were administered. Stomach tightness, nausea, and restless legs were the most common complaints from these patients. None of the patients experienced withdrawal on the second day the about 2 mg buprenorphine together with the about 2 mg flumazenil sublingual tablet was administered.

Pain scores using a VAS did not improve until week 2 with the methadone patients. By week 4, the methadone patients were willing to titrate down and by week 6 there was significant improvement in the VAS and no difference was noted with methadone and other opiate tolerant groups after the sixth (6^{th}) week.

An unexpected enhancement of the opiate analgesic and calming effect of buprenorphine was noted in patients withdrawing from methadone. These patients historically have a prolonged sub-acute withdrawal from methadone but inclusion of the flumazenil in the pharmaceutical dosage unit that also comprised the buprenorphine noticeably enhanced the pain and withdrawal relieving effect of the buprenorphine which was unexpected.

### EXAMPLE 3

In Example 3 a method (protocol) was developed to detox benzodiazepine patients. Patients had been taking a combination of opiates with various benzodiazepines chronically (benzodiazepine dependent patients), and thus were likely addicted to such benzodiazepines. Prior to administering embodiments of the invention comprising the benzodiazepine blocker, such as flumazenil, such benzodiazepine addicted patients were first withdrawn from the benzodiazepine. Otherwise administering embodiments of the invention comprising the benzodiazepine blocker, such as flumazenil, to a still presently non-withdrawn benzodiazepine user may cause adverse effects. For example, such adverse effects may be the patient having seizures and/or panic attacks. For this reason, a method was developed to detox benzodiazepine patients.

Benzodiazepines that were being taken prior to initiating therapy were discontinued 6 days before the first treatment dose of a pharmaceutical dosage unit comprising a buprenorphine and flumazenil combination. Urine screens became negative for benzodiazepines prior to administering the pharmaceutical dosage unit comprising the buprenorphine and the flumazenil combination within this time frame. The majority of patients required phenobarbital or baclofen to help with acute benzodiazepine withdrawal symptoms for an average of 9 days. Once, urine screens became negative for benzodiazepines, various administrations of various pharmaceutical dosage units comprising the buprenorphine and the flumazenil were administered to these patients. Adding an oral treatment option rather than sublingual allowed patients not physically dependent on opiates to participate as nearly all buprenorphine was converted to norbuprenorphine by the liver leaving only flumazenil for binding exclusively to the benzodiazepine receptor. No buprenorphine effects were noted by the orally dosed patients. Patients that were physically dependent on both benzodiazepines and opiates were dosed sublingually. The patients were followed for 12 weeks and no patients restarted benzodiazepines or experienced tremors, nystagmus of physical signs attributed to benzodiazepine withdrawal during the observed period.

### EXAMPLE 4

Subjects were pretreated with about 10 mg flumazenil orally. After 30 minutes midazolam (a benzodiazepine) 0.3 mg was administered and repeated every 30 minutes until sedative effects were noticed by the subject or reported by the investigator. Oral flumazenil of about 10 mg was able to block midazolam for an average of 150 minutes.

Once flumazenil cleared the serum (e.g., in about 3 hours on average) onset of sedation was instantaneous (i.e., in 1 to 5 minutes), usually maximum sedation was achieved 5 minutes after IV midazolam dosing and the sedation began to clear in all subjects by 15 to 20 minutes.

This example study was repeated with about 30 mg flumazenil orally (2 subjects) and sublingually (3 subjects). Allowing the sublingual flumazenil treatment option was thought to avoid first pass hepatic metabolism and potentially increase Cmax (maximum [or peak] serum concentration), shorten Tmax (time at which the Cmax is observed) and is expected to increase the area under the curve for flumazenil in the serum. In this dose range study subjects were again pretreated with about 30 mg flumazenil orally or sublingually. After 30 minutes midazolam (Versed) 0.3 mg was administered and repeated every 30 minutes until sedative effects were noticed by the subject or reported by the investigator. Flumazenil of about 30 mg was able to block midazolam for an average of 255 minutes (240 and 270 minutes) in orally administered subjects and sublingual of about 30 mg flumazenil reproducibly blocked sedation for an average of 300 minutes. All subjects had measurable (but reduced) sedation at the 330 minute interval.

Flumazenil may have an elimination half-life of 84 minutes. By increasing the oral dose of flumazenil from about 10 mg to about 30 mg, the serum level may be maintained inside the therapeutic range for an extended period of time; e.g., 150 minutes for oral about 10 mg flumazenil versus 240 minutes for about 30 mg flumazenil. The Tmax of most short acting opiates (such as, but not limited to, oxycodone) and most orally administered benzodiazepines (such as, but not limited to, midazolam) is in the 1 to 3 hour range with an onset of action before thirty minutes. That means both the about 10 mg flumazenil (150 minutes of protection) and the about 30 mg flumazenil dose (240 minutes of protection) may offer significant protection from overdose from opiates, benzodiazepines and their combination use.

We believe an important factor may be fear of adverse events in those patients that are illegally taking diverted medications including benzodiazepines. Individuals diverting medications rarely, if ever, take a drug history and our disclosed formulations (comprising one or more opioid analgesics present in therapeutically effective amounts and a benzodiazepine blocker present in therapeutically effective amounts) and methods of treating conditions using such disclosed formulations cannot be successfully diverted and used by patients taking opiates and benzodiazepine together. Such combinations of opiates and benzodiazepines makes up a significant portion of the diverted drug use in America (e.g., over 50%) and an even larger fraction of fatal overdoses (e.g., most are polysubstance), with both benzodiazepines and opiates often being present in such fatal overdoses. Our formulations and methods of treating conditions are unique in this diversion deterrence logic and scheme. It uniquely has abuse deterrence and diversion deterrence and are the only formulations and methods of treating conditions that logically may simultaneously address both abuse deterrence and diversion.

In summarizing Example 4: About 3 mg IV flumazenil had 60 minutes blockade from Versed (a midazolam benzodiazepine sedative). About 10 mg oral flumazenil had 150 minutes of protection from the sedation associated with benzodiazepines. (That is, blocking the sedation of Versed). About 30 mg oral flumazenil had a minimum of 240 minutes of protection from the sedation associated with benzodiazepines. About 30 mg flumazenil had up to 300 minutes of protection from sedation when administered sublingually which may be a preferred administration pathway for flumazenil combinations with an opioid analgesic, such as buprenorphine, and the recommended dosing form of this formulation. Additionally, the flumazenil benzodiazepine blockage trends from about 3 mg, about 10 mg, to about 30 mg, supports that trend to continue up to and including about 61 mg of flumazenil.

Various pharmaceutical dosage units and various methods of treatment utilizing the various pharmaceutical dosage units have been described and disclosed. The foregoing description of the various exemplary embodiments of the invention has been presented for the purposes of illustration and disclosure. It is not intended to be exhaustive or to limit the invention to the precise form disclosed. Many modifications and variations are possible in light of the above teaching without departing from the spirit of the invention.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

What is claimed is:

1. A method for treating a condition in a human patient comprising a step of administering to the human patient a pharmaceutical dosage unit, wherein the pharmaceutical dosage unit comprises an opioid analgesic in a first therapeutically effective amount and a benzodiazepine blocker in a second therapeutically effective amount.

2. The method for treating the condition in the human patient according to claim 1, wherein the condition is selected from one or more of the group consisting of: pain, chronic pain, subacute pain, chronic and sub-acute pain, anxiety, addiction, opiate addiction, opiate dependence, benzodiazepine addiction, benzodiazepine dependence, opiate and benzodiazepine addiction, unintended over sedation, and respiratory arrest.

3. The method for treating the condition in the human patient according to claim 1, wherein the opioid analgesic is present in a range of about 0.1 to 61 milligrams; and wherein the benzodiazepine blocker is present in a range of about 0.15 to 61 milligrams.

4. The method for treating the condition in the human patient according to claim 1, wherein the opioid analgesic is buprenorphine and the benzodiazepine blocker is flumazenil.

5. The method for treating the condition in the human patient according to claim 4, wherein the buprenorphine is present in a range of about 6 to 10 milligrams; and wherein the flumazenil is present at about 2 milligrams.

6. The method for treating the condition in the human patient according to claim 4, wherein the pharmaceutical dosage unit is administered several times daily to the human patient.

7. The method for treating the condition in the human patient according to claim 4, wherein the buprenorphine is present in an amount of about 4 milligrams.

8. The method for treating the condition in the human patient according to claim 4, wherein the buprenorphine is present in a range of 12 to 30 milligrams; and wherein the flumazenil is present in a range of 2 to 60 milligrams.

9. The method for treating the condition in the human patient according to claim 8, wherein the pharmaceutical dosage unit is administered at least daily to the human patient.

10. The method for treating the condition in the human patient according to claim 4, wherein the buprenorphine is present in an amount of about 4 milligrams; and wherein the flumazenil is present at about 4.5 milligrams.

11. The method for treating the condition in the human patient according to claim 10, wherein the pharmaceutical dosage unit is administered twice daily to the human patient.

12. The method for treating the condition in the human patient according to claim 1, wherein the benzodiazepine blocker is present in an amount of about 2 to 60 milligrams.

13. The method for treating the condition in the human patient according to claim 1, wherein the benzodiazepine blocker is Flumazenil.

14. The method for treating the condition in the human patient according to claim 1, wherein the opioid analgesic is present in an amount of about 2 to 61 milligrams.

15. The method for treating the condition in the human patient according to claim 1, wherein the opioid analgesic is buprenorphine.

16. The method for treating the condition in the human patient according to claim 1, wherein the opioid analgesic is selected from one or more of the group consisting of: alfentanil, allylprodine, alphaprodine, anileridine, benzylmorphine, bezitramide, bromadol, buprenorphine, butorphanol, carfentanil, clonitazene, codeine, cyclazocine, desomorphine, dextromoramide, dextropropoxyphene, dezocine, diampromide, diamorphine (heroin), dihydrocodeine, dihydroetorphine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, fentanyl, heroin, hydrocodone, hydromorphone, hydroxypethidine, isomethadone, ketobemidone, levallorphan, levorphanol, levophenacyl morphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papavereturn, pentazocine, pethidine (meperidine hydrochloride), phenadoxone, phenomorphan, phenazocine, phenoperidine, piminodine, piritramide, propheptazine, promedol, properidine, propiram, propoxyphene, sufentanil, tapentadol, tilidine, tramadol, salts thereof, and acids thereof.

17. The method for treating the condition in the human patient according to claim 1, wherein the opioid analgesic is hydromorphone and the benzodiazepine blocker is flumazenil; wherein the hydromorphone is present in an amount of about ½ to about 9 milligrams; and wherein the flumazenil is present in an amount of about 2 milligrams.

18. The method for treating the condition in the human patient according to claim 1, wherein the opioid analgesic is hydrocodone and the benzodiazepine blocker is flumazenil; wherein the hydrocodone is present in an amount of about 2.5 to about 31 milligrams; and wherein the flumazenil is present in an amount of about 2 to about 24 milligrams.

19. The method for treating the condition in the human patient according to claim 1, wherein the opioid analgesic is oxycodone and the benzodiazepine blocker is flumazenil; wherein the oxycodone is present in an amount of about 2.5 to about 31 milligrams; and wherein the flumazenil is present in an amount of about 2 to about 24 milligrams.

20. The method for treating the condition in the human patient according to claim 1, wherein the opioid analgesic is morphine and the benzodiazepine blocker is flumazenil; wherein the morphine is present in an amount of about 9 to about 41 milligrams; and wherein the flumazenil is present in an amount of about 2 to about 24 milligrams.

21. The method for treating the condition in the human patient according to claim 1, wherein the opioid analgesic is methadone and the benzodiazepine blocker is flumazenil; wherein the methadone is present in an amount of about 10 to 20 milligrams per milliliter of the pharmaceutical dosage unit; and wherein the Flumazenil is present in an amount of about ¼ to about 10 milligram per milliliter of the pharmaceutical dosage unit.

22. The method for treating the condition in the human patient according to claim 1, wherein the opioid analgesic is buprenorphine and the benzodiazepine blocker is flumazenil; wherein the buprenorphine is present in an amount of about 2 milligrams to about 8 milligrams; and wherein the flumazenil is present in an amount of about 2 milligrams.

23. The method for treating the condition in the human patient according to claim 1, wherein the opioid analgesic is buprenorphine and the benzodiazepine blocker is flumazenil; wherein the buprenorphine is present in an amount of about 2 milligrams to about 8 milligrams; and wherein the flumazenil is present in an amount of about 2.5 milligrams.

24. The method for treating the condition in the human patient according to claim 23, wherein the pharmaceutical dosage unit is administered three times daily to the human patient.

25. The method for treating the condition in the human patient according to claim 1, wherein the opioid analgesic is codeine and the benzodiazepine blocker is flumazenil; wherein the codeine is present in an amount of about 14 to about 61 milligrams; and wherein the flumazenil is present in an amount of about 2 to about 14 milligrams.

26. The method for treating the condition in the human patient according to claim 1; wherein the pharmaceutical dosage unit is a tablet; wherein the opioid analgesic is buprenorphine in an amount of about 4 milligrams per tablet; wherein the benzodiazepine blocker is flumazenil in an amount of about of 2 milligrams per tablet; wherein the tablet further comprises Sugar Base A.

27. The method for treating the condition in the human patient according to claim 26; wherein the Sugar Base A comprises: DiPac at about 77 percent by weight by per tablet, lactose at about 7 percent by weight by per tablet, microcrystalline cellulose at about 5 percent by weight by per tablet, flavoring at about 5 percent by weight by per tablet, crospovidone at about 5 percent by weight by per tablet, and magnesium stearate at about 1 percent by weight per tablet.

28. The method for treating the condition in the human patient according to claim 26; wherein each tablet comprises Coloring A, wherein Coloring A is added in about 20 milligrams of Coloring A per 150 tablets.

29. The method for treating the condition in the human patient according to claim 1; wherein the pharmaceutical dosage unit further comprises an opioid antagonist.

30. The method for treating the condition in the human patient according to claim 29; wherein the opioid antagonist is naloxone; wherein the naloxone is present in amount of about 0.2 milligrams.

31. The method for treating the condition in the human patient according to claim 1; wherein the pharmaceutical dosage unit is a tablet; wherein the opioid analgesic is buprenorphine in an amount of about 4 milligrams per tablet; wherein the benzodiazepine blocker is flumazenil in an amount of about of 2 milligrams per tablet; wherein the tablet further comprises naloxone in an about of 0.2 to about 1 milligrams per tablet; wherein the table further comprises Sugar Base A.

32. The method for treating the condition in the human patient according to claim 31; wherein the Sugar Base A comprises: DiPac at about 77 percent by weight by per tablet, lactose at about 7 percent by weight by per tablet, microcrystalline cellulose at about 5 percent by weight by per tablet, flavoring at about 5 percent by weight by per tablet, crospovidone at about 5 percent by weight by per tablet, and magnesium stearate at about 1 percent by weight per tablet.

33. The method for treating the condition in the human patient according to claim 31; wherein each tablet comprises Coloring A, wherein Coloring A is added in about 20 milligrams of Coloring A per 150 tablets.

34. The method for treating the condition in the human patient according to claim 1, wherein the pharmaceutical dosage unit is administered by a delivery method selected from one or more of the group of: sublingual, mucosal, parenteral, intravenous, intrathecal, subcutaneous, intramuscular, and transdermal delivery methods.

35. The method for treating the condition in the human patient according to claim 1, wherein the benzodiazepine blocker is flumazenil; wherein the presence of the flumazenil in the pharmaceutical dosage unit reduces risks associated with the patient having taken a combination of at least one opiate with at least one benzodiazepine; wherein such risks are selected from one or more of the following group consisting of: unintended over sedation, respiratory arrest, and death; wherein the condition comprises at least the unintended over sedation or the respiratory arrest.

36. The method for treating the condition in the human patient according to claim 1, wherein the method is also for minimizing drug diversion.

37. The method for treating the condition in the human patient according to claim 1, wherein the opioid analgesic is a partial opiate agonist that reduces drug tolerance; wherein the drug tolerance is with respect to at least one opiate.

38. The method for treating the condition in the human patient according to claim 37, wherein the partial opiate agonist is buprenorphine.

39. The method for treating the condition in the human patient according to claim 1, wherein the opioid analgesic is a partial opiate agonist that reduces a severity of opiate withdrawal symptoms; wherein opiate withdrawal symptoms are selected from one or more of the group consisting of: sweating, muscle cramps, physical restlessness, muscle jerks, pupil dilation, bone and/or joint aches, rhinorrhea, lacrimation, nausea, vomiting, diarrhea, tremors, shaking, recurrent yawning, anxiety, irritability, and hair piloerection.

40. The method for treating the condition in the human patient according to claim 39, wherein the partial opiate agonist is buprenorphine.

41. The method for treating the condition in the human patient according to claim 1, wherein the opioid analgesic is a partial opiate agonist that reduces opiate side effects in the patient.

42. The method for treating the condition in the human patient according to claim 41, wherein the opiate side effects are selected from one or more of the group consisting of: sedation, headaches, dizziness, nausea, constipation, dyscoordination, and liver toxicity.
